(19) United States
(12) Patent Application Publication
(10) Pub. No.: US 2002/0120952 A1
(43) Pub. Date: Aug. 29, 2002

(54) Liquid reagent for calcium assay

(76) Inventors: Hirokazu Matsukawa, Osaka-fu (JP); Osamu Oka, Kyoto-fu (JP); Tuyosi Fujita, Osaka-fu (JP); Hitoshi Sakakibara, Kyoto-fu (JP); Tatsuo Sugiyama, Aichi-ken (JP)

Correspondence Address: BURNS, DOANE, SWECKER & MATHIS, L.L.P., P.O. Box 1404, Alexandria, VA, 22313-1404, US

(21) Appl. No.: 09/734,731

(22) Filed: Dec. 13, 2000

Related U.S. Application Data
(62) Division of application No. 09/361,987, filed on Jul. 28, 1999, now abandoned.

(30) Foreign Application Priority Data
Jul. 30, 1998 (JP) 10-228565
May 25, 1999 (JP) 11-145173

(51) Int. Cl. A01H 1/00

(52) U.S. Cl. 800/278

(57) ABSTRACT

The present invention relates to an enzyme immunoassay method for assaying calcium ion, including putting calcium-dependent glutamate dehydrogenase, namely glutamate dehydrogenase (GLDH) never expressing its activity in the absence of calcium ion, in contact to the calcium ion in a sample to assay the enzyme activity of the GLDH, the activity changing depending on the calcium ion concentration in the sample; and the invention also relates to an assay reagent for use in the same. Because the reagent is very stable in a solution state under storage, the reagent can be incorporated in a liquid reagent. Thus, the calcium in a biological material or other samples can be assayed in a simple and accurate manner.

### DETAILED DESCRIPTION OF THE INVENTION

1. Technical Field to which the Invention Belongs

The present invention relates to a liquid reagent for assaying calcium derived from a biological material in a sample. Characteristically, the inventive reagent contains calcium-dependent glutamate dehydrogenase and an adjusting factor and is in a liquid form.

Additionally, the invention relates to a method for assaying calcium in a biological material by using the assay reagent, and a system for efficiently producing calcium-dependent glutamate dehydrogenase.

2. Prior Art

Calcium ion plays a significant role in biological organisms. In biological organisms, 99% of calcium is locally present in bone and teeth, but calcium is continuously absorbed in bone or excreted therefrom at 700 mg/day. Thus, calcium is also present in body fluids and cells at a lesser amount than the amount in bone. The calcium in body fluids and cells is responsible for significant functions as second messenger in biological actions, including nerve transmission function, muscle contraction function, and hormonal actions, in addition to blood coagulation. Thus, the calcium level in body fluids, particularly in blood, should be retained constantly in a strict manner. It has been known that blood calcium level is retained constantly by vitamin D, parathyroid hormone and calcitonin; in normal individuals, for example, calcium is present at 9 to 11 mg/dl in blood and the intra-day variation of the calcium level should be strictly retained within ±3% at most. The calcium level varies in response to diseased conditions. Hypercalcemia occurs in diseases such as myxedema, malignant tumor sarcoidosis, hyperproteinemia, and hyperthyroidism; hypocalcemia emerges in diseases such as hypoparathyroidism, osteomalacia, renal rickets, uremia, hypoproteinemia, and bone metastasis of malignant tumor. Even a slight change of the blood calcium level in normal individuals simply indicates that the change is due to a disease. Thus, the blood calcium assay is a very important test item for laboratory tests.

As has been described above, the development of an excellent method for accurately assaying calcium in a simple manner has been expected, while many of assay reagent compositions in freeze-dried powder for laboratory tests are likely to be replaced with assay reagent compositions in solutions. This is because laborious works to dissolve powdery assay reagent compositions in liquids just prior to every use can thereby be skipped at clinical test practice to reduce the burdensome works on the site. The development of a simple liquid reagent for calcium assay is also demanded. From the standpoint of the stability of calcium assay reagents against problematic accidental temperature change under storage, in particular, a calcium assay reagent suitable for long-term storage is demanded.

### PROBLEMS THAT THE INVENTION IS TO SOLVE

The invention has been achieved so as to satisfy such demands in the industry. It is an object of the invention to develop a novel calcium assay liquid reagent storable for a long term despite the liquid form.

### BRIEF DESCRIPTION OF THE DRAWINGS

FIG. 1 depicts the mechanism of GLDH enzyme reaction;

FIG. 2 depicts the mechanism of calcium assay by GLDH;

FIG. 3 depicts the responsiveness of the concentration-dependent activity within the serum calcium concentration range; and

FIG. 4 is a graph of a calcium standard curve with NTA as the adjusting factor.

### MEANS FOR SOLVING THE PROBLEMS

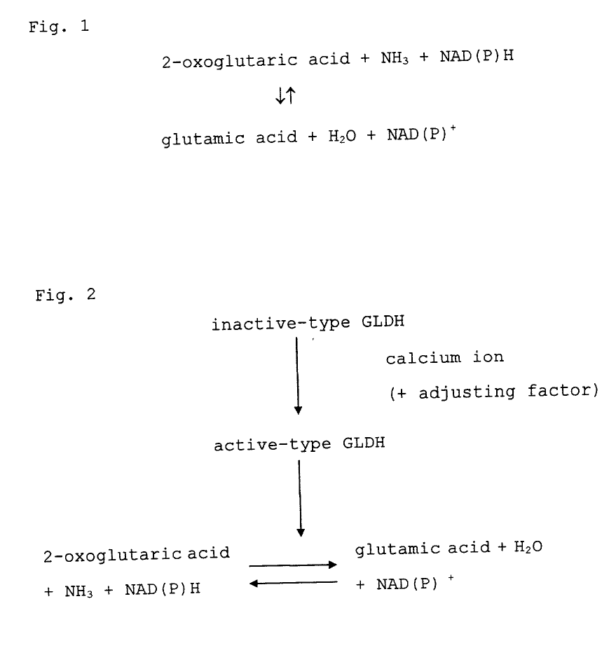

The aforementioned object is attained by the invention. The inventors have made investigations from the standpoint of a wide variety of fields. The inventors have concentrated their attention to glutamate dehydrogenase (referred to as GLDH hereinafter). Generally, GLDH is an enzyme catalyzing the reaction represented by the formula 1 in FIG. 1, including for example enzymes represented by Enzyme Nos. EC 1.4.1.2., EC 1.4.1.3. and EC 1.4.1.4.

Among them, the enzyme of the Enzyme No. EC 1.4.1.2. specific to NAD³⁰ (nicotinamide adenine dinucleotide; at oxidized state) is distributed in animal tissues, plants and bacteria. The enzyme of the Enzyme No. EC 1.4.1.3. is reactive with both of NAD⁺ and NADP⁺ (nicotinamide adenine dinucleotide phosphate; at oxidized state) and is locally present in animal liver and renal mitochondria. The enzyme of the Enzyme No. EC 1.4.1.4. present in yeast and bacteria is specific to NADP⁺.

The inventors have further promoted investigations about GLDH. The inventors have focused attention particularly to a GLDH species with no activity exertion unless calcium ion is present (namely, calcium-dependent GLDH) among these GLDH species. Then, the inventors have attempted to contact the calcium-dependent enzyme (GLDH) to the calcium ion in a sample. Consequently, the inventors have first found that the GLDH enzyme activity varies in the presence of a substrate, depending on the calcium ion concentration in a sample and that the assay of the activity enables the assay of calcium ion. After further investigations, consequently, the inventors have newly found that the calcium-dependent GLDH is stable in a liquid phase against temperature change under storage and can therefore retain a high enzyme activity in a solution for a long term. The invention has been achieved when the inventors have selected the calcium-dependent GLDH for a calcium assay system (in a liquid phase) for biological materials. Based on these new and useful findings, the invention has been achieved. In accordance with the invention, characteristically, the calcium-dependent GLDH is used for a liquid reagent for assaying calcium derived from a biological material in a sample.

In accordance with the assay method of calcium in biological materials, the activity of the GLDH is utilized.

More specifically, inactive-type GLDH is converted to active-type GLDH by utilizing calcium ion present in samples such as blood, serum, plasma and pancreatic juice. Then, ammonia, 2-oxoglutaric acid, NAD(P)H and the active-type GLDH are allowed to react together. By subsequently assaying for example the rate of NAD(P)H decrease on the basis of the absorbance around 340 nm, the amount of calcium derived from the biological material is assayed. Through the reaction represented by the formula 2 in FIG. 2, for example, the amount of calcium can be determined.

(Calcium-dependent GLDH)

In accordance with the invention, calcium-dependent GLDH is utilized as shown in the aforementioned reaction. Any calcium-dependent GLDH can be utilized as long as the GLDH can never exert the activity in the absence of calcium ion. Calcium-dependent GLDH derived from plants, particularly maize, is preferable, but with no specific limitation. Maize-derived GLDH can retain the enzyme activity even after thermal treatment under conditions of 70° C. and pH 7.0 for 20 minutes. A liquid reagent for calcium assay can be provided, by using therein an enzyme stable in a solution state under storage.

The amino acid sequence of the calcium-dependent GLDH derived from maize and the nucleotide sequence of the gene encoding the same are described in Sakakibara, H. et al., “Isolation and characterization of a cDNA that encodes maize glutamate dehydrogenase”, Plant Cell Physiol. 36, 5: 789-797, 1995.

The amino acid sequence is shown as SEQ ID No. 1

Other calcium-dependent GLDH from plants, for example green grass (aokusa) or Pisum, can also be utilized.

The inventive calcium-dependent GLDH can be extracted and purified from plants by known methods including for example the method for the maize-derived enzyme as described in Plant Cell Physiol. 36, 5: 789-797, 1995, but with no specific limitation. The inventive calcium-dependent GLDH is satisfactorily a synthetic GLDH or a recombinant GLDH recovered by genetic engineering. A person skilled in the art can readily recover the calcium-dependent GLDH, according to the disclosure of the present Specification.

As described above, the amino acid sequence of the calcium-dependent GLDH and the nucleotide sequence of the gene encoding the calcium-dependent GLDH have been elucidated by the inventors; and therefore, such GLDH can be not only extracted from natural products such as maize and other plants and animals but also synthetically prepared or industrially prepared in a genetic engineering manner by inserting a DNA fragment of the GLDH gene in a known expression vector such as commercially available expression vectors to prepare a plasmid, transforming a host such as Escherichia coli by using the resulting plasmid, and culturing the transformant to recover the objective GLDH.

(Liquid reagent containing calcium-dependent GLDH)

The inventive liquid reagent characteristically contains the calcium-dependent GLDH in an aqueous medium. The calcium-dependent GLDH is contained at a concentration of 0.1 U/ml to 50 U/ml, preferably 0.5 U/ml to 10 U/ml. As to the GLDH unit, herein, one unit of the enzyme is defined as the amount thereof to generate 1 μmol NAD⁺ under standard GLDH assay conditions, for example at 37° C. and pH 8.0, per one minute.

If desired, the inventive liquid reagent contains 0.2 mM to 0.5 mM NADH, preferably 0.3 mM to 0.4 mM NADH and 0.1 mM to 20 mM 2-oxoglutaric acid, preferably 0.5 mM to 10 mM 2-oxoglutaric acid. Satisfactorily, these are preliminarily contained, together with the calcium-dependent GLDH, in the liquid reagent; otherwise, these are charged in separated containers so that these are added at the time of reaction.

In accordance with the invention, furthermore, adjusting factors may satisfactorily be contained in the inventive liquid reagent. In accordance with the invention, as described above, calcium can be assayed satisfactorily by using the calcium-dependent GLDH. It is firstly confirmed that the enzymological examination of the apparent Km of the glutamate dehydrogenase for calcium is 10 μM and that a linear standard curve within a range of 0 to 4 mM Ca, which is significant for serum calcium assay, can be prepared by adjusting the activity of the glutamate dehydrogenase to enable the determination of serum calcium at a lower level in a linear manner.

Factors capable of adjusting the activity of the enzyme for calcium concentration, including chelating agents and others, can be used satisfactorily. Known chelating agents are appropriately used, including EDTA (ethylenediaminetetraacetic acid), CyDTA (trans-1,2-cyclohexanediamine-N,N,N′,N′-tetraacetic acid), DTPA (diethylenetriaminepentaacetic acid), GEDTA (glycol ether diaminetetraacetic acid), TTHA (triethylenetetraminehexaacetic acid), and methyl EDTA (diaminopropane tetraacetic acid) and additionally including NTA (nitrilotriacetic acid), EDTA-OH (hydroxyethylenediaminetriacetic acid), IDA (iminodiacetic acid), and HIDA (hydroxyethyliminodiacetic acid). NTA is preferably illustrated as one example of the chelating agents.

In accordance with the invention, the inventive assay reagent satisfactorily contains the adjusting factor described above. In accordance with the invention, an assay kit comprising another container charging therein such chelating agent is provided. In this case, the content of the adjusting factor is 0.1 to 100 mM; the content of NTA is preferably 1 to 50 mM. Depending on the type of the chelating agents described above, appropriately, the content varies.

(Method for assaying calcium derived from biological material)

Using the inventive reagent, calcium derived from a biological material can be assayed. More specifically, the inventive method uses a single enzyme, namely inactive-type GLDH, which is converted to active-type GLDH, depending on the calcium ion level in the biological material, to assay the enzyme activity of the active-type GLDH to determine the calcium ion level. By concurrently using such adjusting factor, calcium at a lower concentration in a sample can also be assayed.

In accordance with the invention, any sample possibly containing such biological material can be assayed, with no specific limitation, and includes body fluids such as blood, serum, plasma and pancreatic juice. The sample is satisfactorily collected from living organisms or is an artificial preparation such as research reagent.

In accordance with the invention, inactive-type calcium-dependent GLDH is converted to active-type GLDH by using calcium present in a biological material. Subsequently, a reaction solution containing 2-oxoglutaric acid and NADH reacts with the resulting GLDH. The reaction is progressed at pH 6 to 9, preferably at 25° C. to 40° C. for 5 minutes to one hour.

The enzyme activity of the active-type GLDH as converted by the presence of calcium ion can be assayed by known methods. Because NADPH exerts a specific absorption profile at 340 nm, the change of the absorbance at the wave length, as measured prior to and after GLDH reaction, is compared with the data of calcium conversion of GLDH, to determine the calcium level derived from a biological material. The above example is a simple illustration, with no specific limitation.

As has been described above, preferably, a more linear standard curve can be yielded by adding a chelating agent as an adjusting factor to exert its action according to the inventive method (formula 2). In this case, satisfactorily, the adjusting agent composing the inventive liquid reagent for calcium assay can be allowed to exert its action prior to or during calcium conversion of inactive-type GLDH to active-type GLDH, with no specific limitation.

The invention is now described in more detail in the following examples, which never limit the technical scope of the invention. A modification or variation of the invention is readily carried out by a person skilled in the art on the basis of the description of the present Specification, which is also encompassed within the scope of the invention.

### EXAMPLE 1

Calcium-dependent GLDH derived from maize was expressed, extracted and purified as follows.

a. Intrabacterial expression of calcium-dependent GLDH derived from maize

The full-length calcium-dependent GLDH gene from maize is described and reported in the following reference. Sakakibara, H. et al., “Isolation and characterization of a cDNA that encodes maize glutamate dehydrogenase”, Plant Cell Physiol. 36, 5: 789-797, 1995.

A 1.5-kb DNA fragment containing the calcium-dependent GLDH gene and ranging from the restriction BspHI site to the restriction EcoRI site in the full-length region was inserted in the multicloning NcoI/EcoRI site of a commercially available expression vector pTrc99A (Pharmacia Co.), to construct a plasmid pTrcZmGLDH expressing maize glutamate dehydrogenase. A transformant was recovered in a host Escherichia coli (abbreviated sometimes as E. coli) strain JM105. The transformant E. coli JM105/pTrcZmGLDH was cultured in an LB culture medium (0.5% yeast extract, 1% tryptone, and 1% sodium chloride, pH 7.5) containing 50 μg/ml sodium ampicillin, and when the transformant reached its logarithmic growth phase, an expression inducer of recombinant glutamate dehydrogenase, namely 1 mM IPTG (isopropylthio-β-galactoside), was added to the culture. The transformant was recovered at a wet weight yield of 6 g, by shaking culture in a flask containing the culture broth of 2 liters. Glutamate dehydrogenase was expressed and accumulated at the total activity of2,348 units in the bacteria. Herein, the enzyme activity of the glutamate dehydrogenase was assayed under the following conditions according to the aforementioned formula 2.

As illustrated above, in accordance with the invention, GLDH can efficiently be produced by using the transformant Escherichia coli strain prepared by inserting a recombinant plasmid containing at least a part of the gene encoding GLDH in the host strain. The transformant is designated Escherichia coli JM105/pTrcZmGLDH and is deposited as Accession No. FERM BP-6705 under the Budapest Treaty in National Institute of Bioscience and Human-Technology, Agency of Industrial Science and Technology, of 1-3, Higashi 1 chome, Tsukuba-shi, Ibaraki-ken, Japan (the deposit date: Apr. 19, 1999).

(Method for assaying thermo-resistant GLDH)

Assay temperature: 37° C.

Assay wave length: 340 nm

Reaction solution pH: 8.0

Substrate mixture solution:

100 mM TEA-HCl, pH 8.0

1 mM CaCl₂

5 mM 2-oxoglutaric acid

0.2 M NH₄Cl

0.3 mM NADH

One unit of the activity can generate 1 μmol NAD⁺ per one minute.

b. Extraction and purification of maize-derived recombinant GLDH

The starting material transformant of 6 g (in wet weight) recovered through the flask culture was subjected to disruption and extraction of the bacteria and thermal treatment thereof and a combination of various chromatographic means, to elevate the purification ratio of the extracted solution of the disrupted bacteria to 165 fold. The specific activity of the purified enzyme sample was 660 U/mg. The purification process is schematically shown in the following Table 1.

TABLE 1

| | Protein | Total activity | Specific activity | Purification |
|---|---|---|---|---|
| Purification process | (mg) | (U) | (U/mg) | ratio |
| Ultrasonic | 590 | 2348 | 4.0 | 1 |
| disruption/enzyme | | | | |
| extraction | | | | |
| Supernatant after | 472 | 2111 | 4.5 | 1.1 |
| thermal treatment at | | | | |
| 70° C. | | | | |
| Ion exchange | 10.6 | 1609 | 152 | 38 |
| chromatography on | | | | |
| DEAE-Toyo pearl column | | | | |
| Hydrophobic | 1.8 | 1200 | 660 | 165 |
| chromatography on | | | | |
| phenyl-Toyo pearl | | | | |
| column | | | | |

Note: Protein content was calculated at E 1%/280nm=10.0.

c. Maize-derived recombinant GLDH activity

The enzyme activity of the maize-derived calcium-dependent GLDH recovered by the extraction and purification process was assayed under the conditions described in Example 1a. Consequently, purified maize-derived GLDH was recovered at a yield of 40,000 units from 40 liters of the bacterial culture.

The resulting calcium-dependent GLDH exerted the following properties.

Molecular weight: 290 kDa (6-mer) by gel filtration

Subunit molecular weight: 44, 091

pH activity: optimum pH 7.0 to 8.0

pH stability: pH 5 to 11 (20-min treatment at 70° C.)

Metal ion activation: Km at about 10 μM for Ca²⁺ (Ca⁺⁺); other metal ions never work to activate the enzyme.

Action temperature: 20° C. to 80° C.

Thermal stability: retaining 80% or more of the initial activity after thermal treatment at 70° C. (at pH 7.0 for 20 minutes)

Stability in solution under storage: retaining 60% or more of the initial activity after one-week accelerated tests at 37° C. despite the addition or no addition of an activating agent calcium ion.

### EXAMPLE 2

Adjustment of enzyme activity for calcium assay

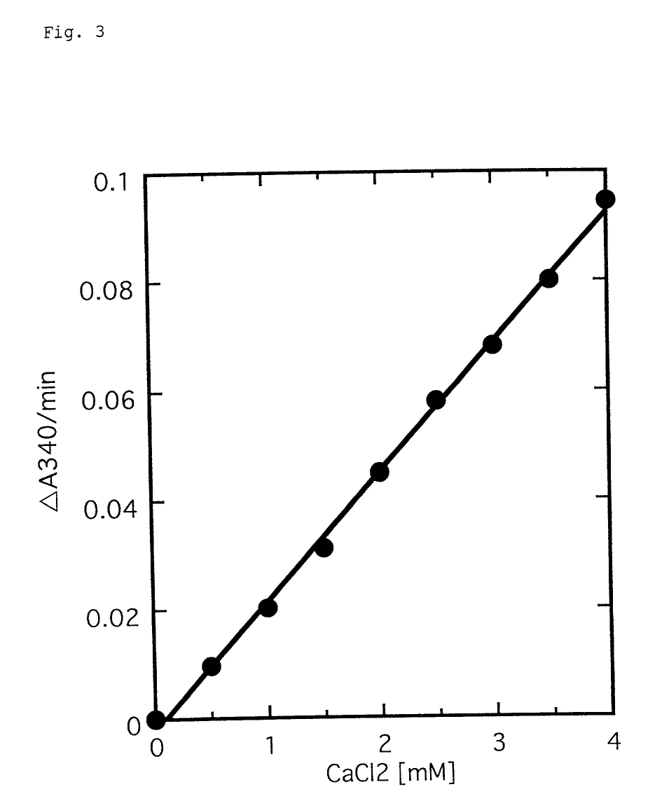

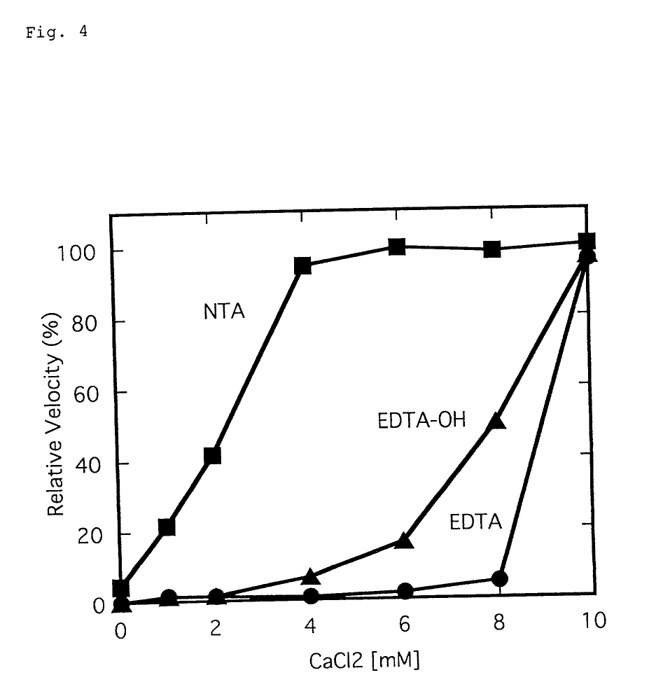

Using the calcium-dependent GLDH recovered in Example 1, assessment was made about the adjustment of the enzyme activity for calcium concentration for assaying serum calcium level. So as to examine the activation in response to the concentration within a range of serum calcium concentration, the activation of maize-derived GLDH by calcium concentration in the presence of various chelating agents added as adjusting factors, each at 0.2 mM, was analyzed in a pattern. On the axis of abscissa is shown the sample calcium concentration; on the axis of ordinate is shown the relative ratio (%) of enzyme activation. The results are shown in FIG. 3. It is indicated that the addition of various chelating agents can adjust the calcium activation. At a 0-4 mM calcium concentration in a sample, NTA is particularly effective as an adjusting factor to permit almost linear activation response.

(Preparation of reaction test solution)

First reagent:

100 mM TEA-HCl buffer, pH 7.5

0.2 M NH₄Cl

10 mM 2-oxoglutaric acid

0.1 U/ml maize-derived GLDH

0.2 mM each of various chelating agents in FIG. 3.

Second reagent:

100 mM TEA-HCl buffer, pH 7.5

0.3 mM NADH

(Calcium sample solution)

Assay apparatus: Automatic analyzer Cobas Fara (manufactured by Roche, Co.)

Assay temperature: 37° C.

Assay wave length: 340 nm

Ratio of added reagents:

sample:pure water:first reagent:second reagent=6:4:240:60 (μl)

Calculation of activation ratio:

Second reagent containing NADH is added to a mixture of a calcium sample solution and the first reagent, to determine the calcium activation pattern based on the initial velocity of enzyme reaction (triangle A340/min).

### EXAMPLE 3

Assay of serum calcium

Based on the results of Example 2, furthermore, the relation between the NTA concentration and the calcium standard curve was examined. It was found that a linear standard curve could be prepared by using an assay reagent prepared as described below. The results are shown in FIG. 4. Additionally, the calcium level in a commercially available serum (Consera as Sample 1; manufactured by Nissui Pharmaceuticals, Co.) was assayed by using a standard curve prepared by using an aqueous calcium chloride solution as the standard solution; the recovery rate of calcium was calculated by using Sample 2 prepared by adding 1 mM calcium chloride to Sample 1. Consequently, as shown in the following Table 2, the recovery rate of calcium added to the Sample 1 from the Sample 2 was calculated to be 100.2%. It was indicated that the serum calcium was specifically assayed by using the prepared reagent.

TABLE 2

| | Assayed calcium | |
|---|---|---|
| Sample | (mmol/liter) | CV (n = 10) |
| Sample 1 | 1.87 | 1.7% |
| (human serum) | | |
| Sample 2 | 2.89 | 2.2% |
| (Sample 1 + 1 mM CaCl₂) | | |

(Preparation of serum calcium assay reagent)

First reagent:

100 mM TEA-HCl buffer, pH 7.5

10 mM 2-oxoglutaric acid

200 MM NH₄Cl

0.3 U/ml maize-derived GLDH

Second reagent:

10 mM NaHCO₃-NaOH buffer, pH 9.0

1.2 mM NADH

20 mM NTA

(Calculation of absorbance change for assaying serum calcium)

Calcium was assayed by the procedures described in Example 2.

### ADVANTAGES OF THE INVENTION

As has been described above, the inventive liquid reagent can retain its high enzyme activity in solution for a long term because of the use of calcium-dependent GLDH therein. The reagent is very stable in a solution state under storage, so the reagent can be incorporated in a liquid reagent kit with ready handling, to enable simple and accurate calcium assay in a biological sample and other samples.

What is claimed is:

1. A liquid reagent for calcium assay, containing calcium-dependent glutamate dehydrogenase.

2. A liquid reagent according to claim 1, wherein the calcium-dependent glutamate dehydrogenase is derived from a plant.

3. A liquid reagent according to any one of claims 1 and 2, wherein the calcium-dependent glutamate dehydrogenase is prepared by using a transformant prepared by inserting an expression plasmid of the enzyme, namely pTrcZmGLDH, in a host.

4. A liquid reagent according to any one of claims 1 to 3, wherein the calcium-dependent glutamate dehydrogenase is of an amino acid sequence of SQ ID No. 1.

5. A liquid reagent according to any one of claims 1 to 4, additionally containing an adjusting factor.

6. A liquid reagent according to claim 5, wherein the adjusting factor is a chelating agent.

7. A method for assaying calcium, comprising using a liquid reagent according to any one of claims 1 to 6.

8. A plasmid pTrcZmGLDH expressing maize-derived calcium-dependent glutamate dehydrogenase.

9. A transformant Escherichia coli JM105/pTrcZmGLDH prepared by inserting an expression plasmid according to claim 8 in a host.

10. A method for preparing calcium-dependent glutamate dehydrogenase, comprising using a transformant according to claim 9.


SEQUENCE LISTING (1 sequence)

SEQ ID NO: 1 - protein, 411 aa, Maize
MNALAATSRNFKQAAKLLGLDSKLEKSLLIPFREIKVECTIPKDDGTLASYVGFRVQHDNARGPMKGGIRYHHEVDPDEVNALAQLMTWKTAVANIPYGGAKGGIGCSPGDLSISELERLTRVFTQKIHDLIGIHTDVPAPDMGTNSQTMAWILDEYSKFHGYSPAVVTGKPVDLGGSLGRDAATGRGVLFATEALLAEHGKGIAGQRFVIQGFGNVGSWAAQLISEAGGKVIAISDVTGAVKNVDGLDIAQLVKHSAENKGIKGFKGGDAIAPDSLLTEECDVLIPAALGGVINKDNANDIKAKYIIEAANHPTDPEADEILSKKGVLILPDILANSGGVTVSYFEWVQNIQGFMWDEEKVNAELRTYITRAFGNVKQMCRSHSCDLRMGAFTLGVNRVARATVLRGWEA